(19) United States
(12) Patent Application Publication
(10) Pub. No.: US 2007/0179152 A1
(43) Pub. Date: Aug. 2, 2007

(54) Combination therapy for the treatment of neoplasms

(76) Inventors: Margaret S. Lee, Middleton, MA (US); M. James Nichols, Boston, MA (US); Amy Beth Wilson, Cambridge, MA (US); Grant R. Zimmermann, Somerville, MA (US)

Correspondence Address: CLARK & ELBING LLP, 101 FEDERAL STREET, BOSTON, MA, 02110, US

(21) Appl. No.: 10/558,136

(22) PCT Filed: May 21, 2004
(86) PCT No.: PCT/US04/16314

§ 371 Date: Jan. 5, 2007

Related U.S. Application Data
(60) Provisional application No. 60/473,207, filed on May 23, 2003.

(51) Int. Cl. A61K 31/496

(52) U.S. Cl. 514/254.01

(57) ABSTRACT

The invention features compositions, methods, and kits for the treatment of neoplasms

### BACKGROUND OF THE INVENTION

The invention relates to the treatment of neoplasms such as cancer.

Cancer is a disease marked by the uncontrolled growth of abnormal cells. Cancer cells have overcome the barriers imposed on normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream, may ensue, destroying healthy tissue.

According to a recent American Cancer Society study, approximately 1,268,000 new cancer cases were expected to be diagnosed in the United States in the year 2001 alone. Lung cancer is the most common cancer-related cause of death among men and women, accounting for over 28% of all cancer-related deaths. It is the second most commonly occurring cancer among men and women; it has been estimated that there were more than 169,000 new cases of lung cancer in the U.S. in the year 2001 and accounting for 13% of all new cancer diagnoses. While the rate of lung cancer cases is declining among men in the U.S., it continues to increase among women. According to the American Cancer Society, an estimated 157,400 Americans were expected to die due to lung cancer in 2001.

Cancers that begin in the lungs are divided into two major types, non-small cell lung cancer and small cell lung cancer, depending on how the cells appear under a microscope. Non-small cell lung cancer (squamous cell carcinoma, adenocarcinoma, and large cell carcinoma) generally spreads to other organs more slowly than does small cell lung cancer. Small cell lung cancer is the less common type, accounting for about 20% of all lung cancer.

Other cancers include brain cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer. These cancers, like lung cancer, are sometimes treated with chemotherapy.

Despite the availability of numerous chemotherapeutic agents, there is still a need for treatment regimens for certain cancers, as well as a general desire for safer, more efficacious chemotherapy regimens.

### SUMMARY OF THE INVENTION

We have discovered that certain triazoles, such as itraconazole, enhance the anti-proliferative activity of antiarrhythmic agents, such as amiodarone, nicardipine, or bepridil. This enhancement of the antiproliferative activity of antiarrhythimic agents by triazoles was measured using HCT116 colon adenocarcinoma cell line, SKMEL-28 human melanoma cells, DU145 human prostate cancer cells, and A549 non-small cell lung carcinoma cells, in a cell viability assay. Structural and functional analogs of antiarrhythmic agents are known and can be used in combination with certain triazoles in the methods of the invention.

Accordingly, the invention features a method for treating a patient who has a neoplasm or a patient who is at risk for developing a neoplasm by administering to the patient an antiarrhythmic agent in combination with a triazole having the formula (I): 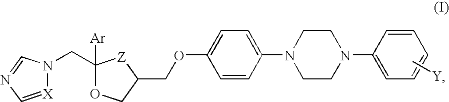
or a pharmaceutically acceptable salt thereof, wherein X is CH₂ or N; Z is CH₂ or O; Ar is selected from the group consisting of phenyl, thienyl, halothienyl, and substituted phenyl having from 1 to 3 substituents, each independently selected from the group consisting of halo, C₁-C₆ linear or branched alkyl, linear or branched C₁-C₆ alkoxy, and trifluoromethyl; and Y is a group having the formula: 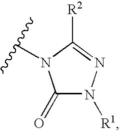
wherein R¹ is selected from the group consisting of C₁-C₆ linear or branched alkyl having 0 or 1 hydroxyl substituents and C₁-C₆ linear or branched alkaryl, and R² is selected from the group consisting of H, linear or branched C₁-C₆ alkyl, and C₁-C₆ alkaryl, wherein said aryl group is a phenyl ring having from 0 to 3 substituents, each independently selected from the group consisting of halo, C₁-C₆ linear or branched alkyl, linear or branched C₁-C₆ alkoxy, and trifluoromethyl. Suitable triazoles of formula (I) include itraconazole, hydroxyitraconazole, posaconazole, and saperconazole.

The compound of formula (I) and the antiarrhythmic agent are administered simultaneously or within 28 days of each other in amounts sufficient to inhibit growth of the neoplasm.

In particular embodiments of the foregoing method, one or both of the administered compounds are approved by a national pharmaceutical regulatory agency, such as the United States Food and Drug Administration (USFDA), for administration to a human. Desirably, the compounds are administered within 14 days of each other, 5 days of each other, within 24 hours of each other, within one hour of each other, or simultaneously. Most desirably, the compounds are administered in the same pharmaceutical formulation, although the compounds can be administered by different routes. Routes of administration include intravenous, intramuscular, subcutaneous, rectal, oral, topical, intravaginal, ophthalmic, or inhalation administration.

The combination of a compound of formula (I) with an antiarrhythmic agent for the treatment of neoplasms allows for the administration of lower doses of each compound, providing similar efficacy or increased efficacy, compared to administration of either compound alone. The methods also allow for the administration of standard doses of each compound, providing improved efficacy, compared to the administration of either compound alone.

A compound of formula (I) and the antiarrhythmic agent are each administered in an amount, frequency, and duration that measurably enhances the effectiveness of the agents to treat a neoplasm. Each of the compound of formula (I) and the antiarrhythmic agent is desirably administered in an amount between 0.1 and 10,000 mg/day, more preferably between 0.1 and 1000 mg/day, and most preferably between 0.1 and 100 mg/day. Alternatively, the compound of formula (1) and/or the antiarrhythmic agent can be administered as a 0.5% to 25% w/v topical formulation. Such topical formulations are particularly useful for treating cancers of the skin and glands of the dermis and epidermis (i.e., sweat glands and sebaceous glands).

The compounds can be provided together in a pharmaceutical composition that contains a pharmaceutically acceptable carrier. Of course, bulk preparations suitable for reformulating into single doses may contain higher amounts. Compounds employed in the methods of the invention can be provided as components of a pharmaceutical pack. Such a pack would typically also include instructions for using the compounds in the methods of the invention. In these packs, compounds can be formulated together or separately and in individual dosage amounts.

The invention also features a method for treating a patient having a neoplasm such as cancer in which the foregoing method is performed in combination with an additional treatment for cancer, such as surgery, radiation therapy, chemotherapy, immunotherapy, anti-angiogenesis therapy, or gene therapy. The two treatments are typically performed within six months of each other, and may even be performed concurrently. Preferably, the additional treatment is chemotherapy. Most preferably, the additional treatment includes administering to a patient cisplatin, daunorubicin, doxorubicin, etoposide, methotrexate, mercaptopurine, 5-fluorouracil, hydroxyurea, vinblastine, vincristine, paclitaxel, bicalutamide, bleomycin, carboplatin, carmustine, cyclophosphamide, docetaxel, epirubicin, gemcitabine hcl, goserelin acetate, imatinib, interferon alpha, irinotecan, lomustine, leuprolide acetate, mitomycin, rituximab, tamoxifen, trastuzumab, or any combination thereof.

Cancers that can be treated according to the method of the invention include leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma. Preferably, the cancer being treated is lung cancer, especially lung cancer attributed to squamous cell carcinoma, adenocarcinoma, or large cell carcinoma, colorectal cancer, ovarian cancer, especially ovarian adenocarcinoma, or prostate cancer.

In particular embodiments of this invention, a compound of formula (I) is administered in combination with an antiarrhythmic agent and one, two, three, or more antiproliferative agents, in amounts and frequencies sufficient to inhibit growth of the neoplasm. Typically, each is administered at least once during a 28-day period, and may, independently, be administered twice, three times, four times, or even daily during that period, as required to inhibit growth of the neoplasm.

The invention also features a method for identifying combinations of compounds useful for treating or preventing a neoplasm in a patient in need of such treatment. The method includes (a) contacting cells in vitro with (i) a triazole or an antiarrhythmic agent and (ii) a candidate compound; and (b) determining whether the combination of the triazole or antiarrhythmic agent and the candidate compound reduces cell proliferation relative to cells contacted with the triazole or antiarrhythmic agent but not contacted with the candidate compound or cells contacted with the candidate compound but not with the triazole or antiarrhythmic agent. A reduction in cell proliferation identifies the combination as one that is useful for treating a patient in need of such treatment.

By “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

By an “antiarrhythmic agent” is meant a drug that reduces cardiac arrhythmia. Examples of antiarrhythmic agents are drugs that block voltage-sensitive sodium channels, beta-adrenoceptor antagonists, drugs that prolong the cardiac action potential, and Ca²⁺ channel antagonists.

By a “triazole” is meant a compound having a five-membered ring of two carbon atoms and three nitrogen atoms. Triazoles that can be employed in the methods of the invention have the formula (I).

By an “antiproliferative agent” is meant a compound that, individually, inhibits the growth of a neoplasm. Antiproliferative agents include, but are not limited to microtubule inhibitors, topoisomerase inhibitors, platins, alkylating agents, and anti-metabolites. Particular antiproliferative agents include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine.

By “inhibits the growth of a neoplasm” is meant measurably slows, stops, or reverses the growth rate of the neoplasm or neoplastic cells in vitro or in vivo. Desirably, a slowing of the growth rate is by at least 20%, 30%, 50%, or even 70%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells, resulting in a shrinkage of the neoplasm.

By “an effective amount” is meant an amount of a compound, alone or in a combination according to the invention, required to inhibit the growth of a neoplasm in vivo. The effective amount of active compound(s) used to practice the present invention for therapeutic treatment of a neoplasm (e.g., cancer) varies depending upon the manner of administration, the age, body weight, and general health of the subject. Ultimately, the attending physician or veterinarian will decide the appropriate amount and dosage regimen. Such amount is referred to as an “effective” amount.

By a “low dosage” is meant at least 10% less than the lowest standard recommended dosage of an antiarrythmic agent or triazole. By a “high dosage” is meant at least 5% more than the highest standard dosage of an antiarrythmic agent or triazole. By a “moderate dosage” is meant the dosage between the low dosage and the high dosage.

As used herein, the terms “alkyl” and the prefix “alk-” are inclusive of both straight chain and branched chain groups and of cyclic groups, i.e., cycloalkyl groups. Cyclic groups can be monocyclic or polycyclic and preferably have from 3 to 20 ring carbon atoms, inclusive. Exemplary cyclic groups are cyclopropyl, cyclopentyl, cyclohexyl, and adamantyl groups.

By “aromatic residue” is meant an aromatic group having a ring system with conjugated π electrons (e.g., phenyl, or imidazole). The ring of the aryl group is preferably 5 to 10 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

The term “aryl” means carbocyclic aromatic rings or ring systems. Examples of aryl groups include phenyl, naphthyl, biphenyl, fluorenyl, and indenyl groups. The term “heteroaryl” means aromatic rings or ring systems that contain at least one ring hetero atom (e.g., O, S, N). Aryl groups may be unsubstituted or substituted by one or more substituents selected from the group consisting of C₁₋₁₀ alkyl, hydroxy, halo, nitro, C₁₋₁₀ alkoxy, C₁₋₁₀ alkylthio, trihalomethyl, C₁₋₁₀ acyl, arylcarbonyl, nitrile, ₁₋₁₀ alkoxycarbonyl, oxo, and arylalkyl (wherein the alkyl group has from 1 to 10 carbon atoms).

By “treating” is meant administering or prescribing a pharmaceutical composition for the treatment or prevention of an inflammatory disease.

By “patient” is meant any animal (e.g., a human).

Compounds useful in the invention include those described herein in any of their pharmaceutically acceptable forms, including isomers such as diastereomers and enantiomers, salts, solvates, and polymorphs, thereof, as well as racemic mixtures and pure isomers of the compounds described herein.

Other features and advantages of the invention will be apparent from the following detailed description, and from the claims.

### DETAILED DESCRIPTION

We have discovered that certain triazole antifimgal agents enhance the antiproliferative activity of compounds used to treat heart arrhythmias against cancer cells in vitro. Thus, a combination of an antiarrhythmic agent with a triazole is useful for the treatment of cancer and other neoplasms.

Antiarrhythmic Agents

Generally, there is little structure-activity relationship between antiarrhythmic agents with regard to their antiarrhythmic effects. By the Vaughan Williams' classification, antiarrhythmic agents are generally divided into four classes.

Class I drugs block voltage-sensitive sodium channels. Class I drugs are further divided into Classes IA, IB and IC. Class IA drugs lengthen the duration of the myocardial action potential while decreasing the maxinal rate of depolarization. Class IA drugs include hydroxyl quinidine, quinidine, disopyramide, and procainamide. Class IB antiarrhythmic agents decrease the maximal rate of depolarization as well as decreasing the duration of the myocardial action potential. Examples of Class IB agents are lidocaine, tocainide, mexiletine, and phenytoin. Class IC antiarrhythmic agents decrease the maximal rate of depolarization while having no effect on the duration of the myocardial action potential. Examples include flecainide and encainide.

Class II drugs are beta-adrenoceptor antagonists, examples of which are propranolol, acebutolol, esmolol, and sotalol.

Class III drugs prolong the cardiac action potential, thereby increasing the refractory period suppressing the ectopic and re-entrant activity, such as amiodarone, sotalol, and bretylium tosylate.

Class IV drugs are Ca²⁺ channel antagonists, which block the slow inward current that is carried by calcium ions during the myocardial action potential. Examples of Class IV drugs are nifedipine, amlodipine, felodipine, flunarizine, isradipine, nicardipine, diltiazem, verapamil, and bepridil.

Other antiarrhythmic agents that do not fall within one of the above categories but are considered antiarrhythmic agents include digoxin and adenosine.

Amiodarone

Amiodarone (2-Butyl-3-benzofuranyl)(4-(2-(diethylamino)ethoxy)-3,5-diidophenyl)methanone; Cordarone™), has the following structure:

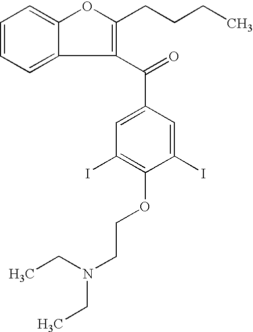

Typically, for treatment of an arrhythmia, arniodarone is administered in an amount according to a patient's condition, but standard recommended dosages between 400 and 1600 mg/day for oral administration or 150-720 mg/day for injection dosage.

Related compounds to amiodarone include di-N-desethylamiodarone, desethylamiodarone, desoxoamiodarone, etabenzarone, and 2-butylbenzofuran-3-yl, 4 hydroxy-3,5-diiodophenyl ketone.

Bepridil

Bepridil (beta-((2-methylpropoxy)methyl)-N-phenyl-N-(phenylmethyl)-1-pyrrolidineethanamine) has the following structure:

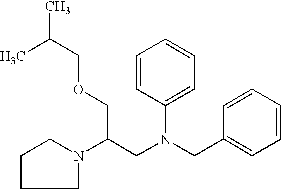

Typically, for treatment of an arrhythmia, bepridil is administered in an amount according to a patient's condition, but a standard recommended dosage is between 200 and 300 mg/day.

Nicardipine

Nicardipine (2-(benzyl-methyl amino)ethyl methyl 1,4-dihydro-2,6-dimethyl-4-(m-nitrophenyl)-3,5-pyridinedicarboxylate monohydrochloride) is a class IV antiarrhythmic having the following structure:

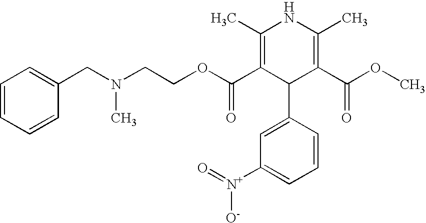

Typically, for treatment of an arrhythmia, nicardipine is administered in an amount according to a patient's condition, but a standard recommended dosage is 20 mg three times per day.

The standard recommended dosage for other antiarrhythmic agents is as follows: amlodipine—5-10 mg/day; nifedipine—10 mg three times per day; diltiazem—90-240 mg/day (in one to three dosages); felodipine—5-10 mg/day; flunarizine—10 mg/day; isradipine, 2.5 mg twice per day; nimodipine—60 mg every four hours; verapamil—40-120 mg three times per day.

Triazoles

Compounds of formula (I) are triazoles, a class of compounds having a five-membered ring of two carbon atoms and three nitrogen atoms. Examples of compounds of formula (I) are itraconazole, hydroxyitraconazole, posaconazole, and saperconazole.

Typically, for treatment of a fungal infection, a triazole is administered in an amount according to a patient's condition, but the standard recommended dosage of itraconazole is between 100 and 400 mg/day.

Formulation of Pharmaceutical Compositions

Suitable modes of administration include oral, rectal, intravenous, intramuscular, subcutaneous, inhalation, topical or transdermal, vaginal, intraperitoneal (IP), intra-articular, and ophthalmic.

Administration of each compound of the combination may be any suitable means that results in a concentration of the compound that, combined with the other component, is anti-neoplastic upon reaching the target region. Compounds are admixed with a suitable carrier substance, and are generally present in an amount of 1-95% by weight of the total weight of the composition. The composition may be provided in a dosage form that is suitable for oral, parenteral (e.g., intravenous, intramuscular, subcutaneous), rectal, transdermal, nasal, vaginal, inhalant, or ocular administration. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds. J. Swarbrick and J. C. Boylan, 1988-2002, Marcel Dekker, N.Y.).

Therapy

The combinations of compounds of the invention are useful for the treatment of neoplasms. Combination therapy may be performed alone or in conjunction with another therapy (e.g., surgery, radiation, chemotherapy, biologic therapy). Additionally, a person having a greater risk of developing a neoplasm (e.g., one who is genetically predisposed or one who previously had a neoplasm) may receive prophylactic treatment to inhibit or delay neoplastic formation. The duration of the combination therapy depends on the type of disease or disorder being treated, the age and condition of the patient, the stage and type of the patient's disease, and how the patient responds to the treatment.

Combination therapy may be provided wherever chemotherapy is performed: at home, the doctor's office, a clinic, a hospital's outpatient department, or a hospital. Treatment generally begins at a hospital so that the doctor can observe the therapy's effects closely and make any adjustments that are needed. The duration of the combination therapy depends on the kind of cancer being treated, the age and condition of the patient, the stage and type of the patient's disease, and how the patient's body responds to the treatment. Drug administration may be performed at different intervals (e.g., daily, weekly, or monthly) and the administration of each agent can be determined individually. Combination therapy may be given in on-and-off cycles that include rest periods so that the patient's body has a chance to build healthy new cells and regain its strength.

Depending on the type of cancer and its stage of development, the combination therapy can be used to treat cancer, to slow the spreading of the cancer, to slow the cancer's growth, to kill or arrest cancer cells that may have spread to other parts of the body from the original tumor, to relieve symptoms caused by the cancer, or to prevent cancer in the first place. Combination therapy can also help people live more comfortably by eliminating cancer cells that cause pain or discomfort.

The dosage, frequency and mode of administration of each component of the combination can be controlled independently. For example, one compound may be administered topically three times per day, while the second compound may be administered orally once per day. Combination therapy may be given in on-and-off cycles that include rest periods so that the patient's body has a chance to recovery from any as yet unforeseen side-effects. The compounds may also be formulated together such that one administration delivers both compounds.

Examples of cancers and other neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

Dosages

The dosage of each compound of the claimed combinations depends on several factors, including: the administration method, the neoplasm to be treated, the severity of the neoplasm, whether the neoplasm is to be treated or prevented, and the age, weight, and health of the, patient to be treated.

A compound of the combination may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubitizer such as ethanol can be applied. One skilled in the art will recognize that if an alternative compound is substituted for either the triazole or antiarrhythmic agent or any one of the antiproliferative agents, the correct dosage can be determined by examining the efficacy of the compound in cell proliferation assays. When used in combination therapy according to the methods of this invention, the antiarrhythmic agent is dosed in an amount and frequency equivalent to or less than those that result in effective anticancer monotherapy using that compound.

The following examples are to illustrate the invention and are not intended to be limiting in any way.

### EXAMPLE 1

### Antiproliferative Activity of Itraconazole and Amiodarone Against Human Colorectal Carcinoma HCT116

The results from a 2-fold dilution series of an amiodarone and itraconazole combination on HCT116 cell growth are shown in Table 1. In the present assay, amiodarone exhibits maximal inhibition of 94.7% at 29 μM, while at 7.3 μM amiodarone shows only 7.4% inhibition. In the presence of 7 μM itraconazole, the efficacy of amiodarone is enhanced, exhibiting 90.5% inhibition with a 4-fold reduction in the concentration of amiodarone.

TABLE 1
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Amiodarone (μM) | 0 | −4.9 | −0.7 | −2.8 | 1.9 | 8.5 | 17.5 | 20.4 | 19.1 | 16.0 |
| | 0.23 | −3.7 | −3.1 | −3.5 | 2.9 | 7.3 | 19.1 | 23.5 | 22.0 | 15.1 |
| | 0.46 | −3.7 | 0.4 | 0.3 | 3.3 | 17.8 | 22.4 | 27.2 | 27.2 | 21.6 |
| | 0.92 | 3.1 | −2.5 | 2.0 | 11.7 | 14.1 | 27.1 | 37.0 | 36.1 | 34.0 |
| | 1.8 | −1.4 | −3.2 | 0.4 | 8.3 | 18.6 | 26.9 | 43.5 | 36.8 | 39.5 |
| | 3.7 | 7.6 | 0.3 | 6.5 | 16.1 | 27.7 | 59.7 | 63.8 | 65.0 | 52.0 |
| | 7.3 | 7.4 | 11.7 | 13.9 | 25.8 | 69.7 | 86.6 | 90.5 | 87.7 | 83.8 |
| | 15 | 36.0 | 46.8 | 53.7 | 84.6 | 92.8 | 94.6 | 95.0 | 95.0 | 94.9 |
| | 29 | 94.7 | 94.5 | 94.8 | 95.0 | 95.3 | 95.6 | 95.6 | 95.5 | 95.4 |

### EXAMPLE 2

### Antiproliferative Activity of Itraconazole and Amiodarone Against Non-Small Cell Lung Carcinoma A549

The results from a 2-fold dilution series of an amiodarone and itraconazole combination on A549 cell growth are shown in Table 2. In the present assay, amiodarone shows practically no inhibition at 7.3 μM. In the presence of 7 μM itraconazole, the efficacy of amiodarone is 77.3%.

TABLE 2
Percent inhibition of Alamar Blue Metabolism in A549 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Amiodarone (μM) | 0 | −0.3 | 2.3 | 3.7 | 11.5 | 2.8 | 8.0 | 6.3 | 7.4 | 9.7 |
| | 0.23 | 2.0 | −0.5 | 3.9 | 11.3 | 3.5 | 11.4 | 11.4 | 7.5 | 10.3 |
| | 0.46 | −1.6 | 1.0 | 3.5 | 2.1 | 5.9 | 10.3 | 10.7 | 10.8 | 10.5 |
| | 0.92 | 11.4 | 1.4 | 8.0 | 3.5 | 4.8 | 21.8 | 15.4 | 14.1 | 12.8 |
| | 1.8 | 3.2 | 4.6 | 0.1 | 8.9 | 4.7 | 19.8 | 20.7 | 17.3 | 16.1 |
| | 3.7 | 2.6 | 11.5 | 6.4 | 8.5 | 10.0 | 40.7 | 38.7 | 34.6 | 26.1 |
| | 7.3 | −3.6 | 1.1 | 2.1 | 3.6 | 22.5 | 70.3 | 77.3 | 55.4 | 54.2 |
| | 15 | 17.3 | 22.5 | 24.6 | 33.8 | 70.5 | 91.5 | 92.3 | 70.3 | 76.1 |
| | 29 | 90.7 | 90.0 | 93.7 | 95.2 | 95.7 | 95.7 | 95.7 | 95.6 | 95.2 |

### EXAMPLE 3

### Antiproliferative Activity of Itraconazole and Amiodarone Against SKMEL-28 Melanoma Cells

The results from a 2-fold dilution series of amiodarone and itraconazole combination on SKMEL-28 cell growth are shown in Table 3. Amiodarone at 7.3 μM shows only 1.4% inhibition, while the combination of 7 pM itraconazole and 7.3 μM amiodarone exhibits 80.8% inhibition.

TABLE 3
Percent inhibition of Alamar Blue Metabolism in SKMEL-28 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Amidoarone (μM) | 0 | 2.8 | 1.3 | 1.5 | 4.4 | 6.6 | 10.4 | 12.6 | 14.0 | 15.0 |
| | 0.23 | 2.2 | 4.4 | 4.5 | 4.6 | 7.7 | 12.1 | 14.2 | 15.6 | 15.0 |
| | 0.46 | 4.2 | 3.1 | 4.2 | 6.5 | 10.5 | 25.1 | 16.3 | 18.5 | 17.7 |
| | 0.92 | 3.8 | 1.5 | 7.0 | 6.3 | 15.9 | 17.6 | 24.8 | 27.2 | 21.0 |
| | 1.8 | 0.8 | −0.6 | 1.2 | 2.8 | 8.4 | 17.7 | 21.4 | 20.5 | 28.0 |
| | 3.7 | −0.3 | −0.9 | 1.4 | 7.3 | 10.4 | 54.6 | 39.1 | 49.4 | 60.6 |
| | 7.3 | 1.4 | 1.8 | 3.0 | 16.0 | 74.8 | 84.4 | 80.8 | 79.8 | 80.7 |
| | 15 | 22.0 | 56.1 | 61.2 | 73.2 | 95.4 | 95.5 | 95.3 | 95.5 | 94.7 |
| | 29 | 96.1 | 96.3 | 96.4 | 96.3 | 96.3 | 96.3 | 96.3 | 96.2 | 96.3 |

### EXAMPLE 4

### Antiproliferative Activity of Amiodarone and Itraconazole Against Humman DU145 Human Prostate Cancer Cells

The results from a 2-fold dilution series of amiodarone and itraconazole combination on DU145 cell growth are shown in Table 4. In the present assay, amiodarone exhibits maximal inhibition of 60.5% at 29 μM, while at 15 μM amiodarone shows practically no inhibition. The combination of 14 μM itraconazole and 15 μM amiodarone exhibits 44.1% inhibition.

TABLE 4
Percent inhibition of Alamar Blue Metabolism in DU145 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Amiodarone (μM) | 0 | −5.1 | −0.6 | −1.6 | 4.9 | −4.2 | −10.1 | −13.0 | −4.3 | −4.6 |
| | 0.23 | −6.0 | −2.9 | 5.7 | 2.4 | −1.0 | −9.6 | −8.7 | −3.4 | 2.3 |
| | 0.46 | −3.0 | 7.7 | 4.9 | 8.1 | −1.7 | −0.6 | −9.0 | 2.0 | 7.2 |
| | 0.92 | 0.4 | 6.5 | 4.6 | 12.6 | 4.9 | −4.8 | −4.3 | 3.0 | 9.4 |
| | 1.8 | −3.1 | 4.6 | 4.4 | 1.3 | −0.2 | −5.8 | −3.6 | 0.3 | 5.1 |
| | 3.7 | 2.3 | 2.6 | −1.2 | 1.2 | −2.4 | −1.9 | −1.7 | 5.0 | 7.8 |
| | 7.3 | −0.8 | 0.1 | −0.4 | −3.7 | −3.4 | 5.1 | 11.1 | 12.6 | 12.8 |
| | 15 | −4.3 | −6.2 | 4.3 | 10.4 | 17.5 | 40.0 | 42.8 | 44.1 | 41.6 |
| | 29 | 50.5 | 50.2 | 58.2 | 62.5 | 72.6 | 71.0 | 83.1 | 82.0 | 84.3 |

### EXAMPLE 5

### Antiproliferative Activity of Bepridil and Itraconazole Against Human Colorectal Carcinoma HCT116

The results from a 2-fold dilution series of bepridil and itraconazole combination on HCT116 cell growth are shown in Table 5. In the present assay, bepridil exhibits maximal inhibition of 96.4% at 48 μM, while at 12 μM bepridil shows only 33.2% inhibition. The combination of 7 μM itraconazole and 12 μM bepridil exhibits 87.3% inhibition.

TABLE 5
Percent inhibition of Alamar Blue Metabolism in HCT116 cells

| | | Bepridil (μM) | | | | | | | | |
|---|---|---|---|---|---|---|---|---|---|---|
| | | 0 | 0.38 | 0.76 | 1.5 | 3 | 6.1 | 12 | 24 | 48 |
| Itraconazole (μM) | 0 | −0.1 | 1.5 | 6.4 | 5.1 | 17.4 | 54.0 | 33.2 | 79.0 | 96.4 |
| | 0.22 | 5.8 | 3.0 | 3.5 | 15.2 | 26.4 | 50.8 | 49.8 | 90.8 | 96.5 |
| | 0.44 | −1.4 | 6.9 | 4.1 | 11.1 | 17.7 | 25.6 | 39.6 | 80.7 | 96.4 |
| | 0.87 | 13.3 | 14.7 | 15.3 | 19.8 | 25.5 | 43.4 | 67.4 | 94.6 | 96.5 |
| | 1.7 | 26.4 | 30.1 | 28.8 | 31.1 | 46.8 | 73.0 | 83.3 | 94.3 | 96.5 |
| | 3.5 | 30.3 | 31.9 | 22.9 | 43.1 | 75.8 | 66.5 | 86.1 | 95.4 | 96.5 |
| | 7 | 33.1 | 23.4 | 33.4 | 29.1 | 43.2 | 74.1 | 87.3 | 94.8 | 96.4 |
| | 14 | 17.8 | 24.7 | 22.8 | 18.5 | 24.3 | 83.5 | 86.7 | 95.2 | 96.5 |
| | 28 | 17.2 | 18.2 | 18.1 | 22.2 | 38.4 | 53.8 | 85.0 | 94.9 | 96.3 |

### EXAMPLE 6

### Antiproliferative Activity of Itraconazole and Nicardipine Against Carcinoma HCT116

The results from a 2-fold dilution series of an amiodarone and itraconazole combination on HCT116 cell growth are shown in Table 6. In the present assay, nicardipine exhibits maximal inhibition of 81.7% at 39 μM, while at 19 μM nicardipine shows only 25.2% inhibition. The combination of 7 μM itraconazole and 19 μM nicardipine exhibits 73.3% inhibition.

TABLE 6
Percent inhibition of Alamar Blue Metabolism in HCT116 cells

| | | Nicardipine (μM) | | | | | | | | |
|---|---|---|---|---|---|---|---|---|---|---|
| | | 0 | 0.3 | 0.61 | 1.2 | 2.4 | 4.8 | 9.7 | 19 | 39 |
| Itraconazole (μM) | 0 | −0.3 | 2.6 | 1.4 | 2.5 | 5.2 | 7.5 | 9.7 | 25.2 | 81.7 |
| | 0.22 | 3.0 | 5.0 | 4.9 | 5.5 | 7.7 | 12.6 | 16.1 | 32.8 | 83.1 |
| | 0.44 | −0.5 | 7.5 | 3.9 | 9.3 | 5.7 | 14.8 | 14.3 | 31.1 | 91.8 |
| | 0.87 | 12.3 | 7.2 | 13.4 | 11.5 | 16.1 | 15.4 | 25.8 | 48.0 | 84.2 |
| | 1.7 | 15.4 | 15.3 | 15.6 | 13.9 | 20.1 | 24.9 | 35.9 | 62.1 | 91.8 |
| | 3.5 | 20.1 | 19.2 | 23.1 | 22.7 | 28.8 | 36.5 | 54.8 | 73.9 | 88.8 |
| | 7 | 32.7 | 18.8 | 23.9 | 26.9 | 31.6 | 37.3 | 49.9 | 73.3 | 88.7 |
| | 14 | 19.8 | 21.1 | 26.7 | 31.2 | 28.3 | 37.6 | 51.6 | 69.1 | 86.4 |
| | 28 | 22.4 | 18.6 | 19.3 | 24.2 | 32.9 | 34.3 | 48.0 | 69.1 | 85.3 |

Materials and Methods

The foregoing results were obtained with the following materials and methods.

Tumor Cell Culture

Human colorectal carcinoma HCT116 (ATCC# CCL-247) cells, non-small cells lung carcinoma A549 (ATCC# CCL-185) cells, human prostate cancer DU145 (ATCC# HTB-81) cells, and human melanoma SKL-28 (ATCC# HTB-72) cells were grown at 37±0.5° C. and 5% CO₂ in RPMI 1640 medium supplemented with 10% FBS, 2 mM glutamine, 1% penicillin, and 1% streptomycin.

Test Compounds

Amiodarone, nicardipine, and bepridil were obtained from Sigma Chemical Co. (St. Louis, Mo.). Itraconazole was obtained from Intrachem (Paramus, N.J.). Stock solutions (1000×) of each compound were prepared in DMSO and stored at −20° C. Master stock plates of 2-fold serial dilutions of individual compounds were prepared in 384-well plates. Combination matrices of test compounds were generated from these master stock plates by dilution into growth media described above. The final concentration of test compounds in the combination matrices was 10× greater than used in the assay. The combination matrices were used immediately and discarded.

Anti-proliferation Assay

The anti-proliferation assays were performed in 384 well plates. The tumor cells were liberated from the culture flask using a solution of 0.25% trypsin. Cells were diluted in culture media such that 3,000 SKMEL cells, or 1,500 cells for all the other cell lines, were delivered in 35 μl of media into each assay well. Assay plates were incubated for 16-24 hours 37° C.±0.5° C. with 5% CO₂. Then, 4.5 μl of 10× stock solutions from the combination matrices were added to 40 μl of culture media. Assay plates were further incubated for 72 hours at 37° C.±0.5° C. with 5% CO₂. Forty microliters of 10.5% Alamar Blue warmed to 37° C.±0.5° C., was added to each assay well following the incubation period. Alamar Blue metabolism was quantified by the amount of fluorescence intensity 3.5-5.0 hours after addition. Quantification, using the LJL Analyst AD reader (LJL Biosystems), was taken in the middle of the well with high attenuation, a 100 msec read time, an excitation filter at 530 nm, and an emission filter at 575 nm. For some experiments, quantification was performed using a Wallac Victor² reader. Measurements were taken at the top of the well with stabilized energy lamp control; a 100 msec read time, an excitation filter at 530 nm, and an emission filter at 590 nm. No significant differences between plate readers were measured.

The percent inhibition (%I) for each well was calculated using the following formula:
%I=[(avg. untreated wells−treated well)/(avg. untreated wells)]×100
The average untreated well value (avg. untreated wells) is the arithmetic mean of 40 wells from the same assay plate treated with vehicle alone. Negative inhibition values result from local variations in treated wells as compared to untreated wells. The data shown in Table 1 are the average of ten 9×9 matrices. The data shown in Table 2 are the average of eight 9×9 matrices. The data shown in Table 4 are the average of six 9×9 matrices. The data shown in Tables 3, 5 and 6 are the average of four 9×9 matrices.

### OTHER EMBODIMENTS

The anti-proliferative effect demonstrated with the tumor cell lines used herein can be similarly demonstrated using other cancer cell lines, such as NSC lung carcinoma, MCF7 mammary adenocarcinoma, PA-1 ovarian teratocarcinoma, HT29 colorectal adenocarcinoma, H1299 large cell carcinoma, U-2 OS osteogenic sarcoma, U-373 MG glioblastoma, Hep-3B hepatocellular carcinoma, BT-549 mammary carcinoma, T-24 bladder cancer, C-33A cervical carcinoma, HT-3 metastatic cervical carcinoma, SiHa squamous cervical carcinoma, CaSki epidermoid cervical carcinoma, NCI-H292 mucoepidermoid lung carcinoma, NCI-2030, non small cell lung carcinoma, HeLa, epithelial cervical adenocarcinoma, KB epithelial mouth carcinoma, HT1080 epithelial fibrosarcoma, Saos-2 epithelial osteogenic sarcoma, PC3 epithelial prostate adenocarcinoma, SW480 colorectal carcinoma, CCL-228, MS-751 epidermoid cervical carcinoma, LOX IMVI melanoma, MALME-3M melanoma, M14 melanoma, SK-MEL-2 melanoma, SK-MEL-28 melanoma, SK-MEL-5 melanoma, UACC-257 melanoma, and UACC-62 melanoma cell lines. The specificity can be tested by using cells such as NHLF lung fibroblasts, NHDF dermal fibroblasts, HMEC mammary epithelial cells, PrEC prostate epithelial cells, HRE renal epithelial cells, NHBE bronchial epithelial cells, CoSmC Colon smooth muscle cells, CoEC colon endothehal cells, NHEK epidermal keratinocytes, and bone marrow cells as control cells.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in oncology or related fields are intended to be within the scope of the invention.

What is claimed is:

1. A pharmaceutical composition, comprising an antiarrhythmic agent; a compound of formula (I): 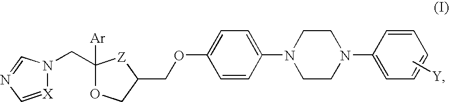
or a pharmaceutically acceptable salt thereof, wherein X is CH₂ or N; Z is CH₂ or O; Ar is selected from the group consisting of phenyl, thienyl, halothienyl, and substituted phenyl, said substituted phenyl having from 1 to 3 substituents, each independently selected from the group consisting of halo, C₁-C₆ linear or branched alkyl, linear or branched C₁-C₆ alkoxy, and trifluoromethyl; and Y is a group having the formula: 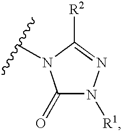
wherein R¹ is selected from the group consisting of C₁-C₆ linear or branched alkyl having 0 or 1 hydroxyl substituents and C₁-C₆ linear or branched alkaryl, and R² is selected from the group consisting of H, linear or branched C₁-C₆ alkyl, and C₁-C₆ alkaryl, wherein said aryl group is a phenyl ring having from 0 to 3 substituents, each independently selected from the group consisting of halo, C₁-C₆ linear or branched alkyl, linear or branched C₁-C₆ alkoxy, and trifluoromethyl; and a pharmaceutically acceptable carrier.

2. The composition of claim 1 wherein the antiarrhythmic agent is amiodarone, di-N-desethylamiodarone, desethylamiodarone, bepridil, or nicardipine.

3. The composition of claim 1, wherein the compound of formula (I) is itraconazole, hydroxyitraconazole, posaconazole, or saperconazole.

4. A method of treating a neoplasm in a patient, said method comprising administering to said patient a pharmaceutical composition of claim 1.

5. The method of claim 4, wherein the neoplasm is selected from the group consisting of colon cancer, lung cancer, non-small cell carcinoma, ovarian cancer, prostate cancer, and leukemia.

6. A method of treating a neoplasm in a patient, said method comprising administering to said patient an antiarrhythmic agent and a compound of formula (I): 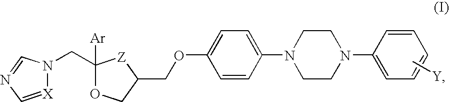
or a pharmaceutically acceptable salt thereof, wherein X is CH₂ or N; Z is CH₂ or O; Ar is selected from the group consisting of phenyl, thienyl, halothienyl, and substituted phenyl, said substituted phenyl having from 1 to 3 substituents, each independently selected from the group consisting of halo, C₁-C₆ linear or branched alkyl, linear or branched C₁-C₆ alkoxy, and trifluoromethyl; and Y is a group having the formula: 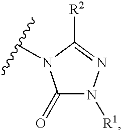
wherein R¹ is selected from the group consisting of C₁-C₆ linear or branched alkyl having 0 or 1 hydroxyl substituents and C₁-C₆ linear or branched alkaryl, and R² is selected from the group consisting of H, linear or branched C₁-C₆ alkyl, and C₁-C₆ alkaryl, wherein said aryl group is a phenyl ring having from 0 to 3 substituents, each independently selected from the group consisting of halo, C₁-C₆ linear or branched alkyl, linear or branched C₁-C₆ alkoxy, and trifluoromethyl, wherein said antiarrhythmic agent and said compound of formula (I) are administered in amounts that together are effective to treat said neoplasm.

7. The method of claim 6, wherein the antiarrhythmic agent is amiodarone, di-N-desethylamiodarone, desethylamiodarone, bepridil, or nicardipine.

8. The method of claim 6, wherein the compound of formula (I) is itraconazole, hydroxyitraconazole, posaconazole, or saperconazole.

9. The method of claim 6, wherein the neoplasm is selected from the group consisting of colon cancer, lung cancer, non-small cell carcinoma, ovarian cancer, prostate cancer, or leukemia.

10. The method of claim 6 wherein the antarrhythmic agent and the compound of formula (I) are administered within 14 days of each other.

11. The method of claim 10, wherein the antarrhythmic agent and the compound of formula (I) are administered within 7 days of each other.

12. The method of claim 11, wherein the antarrhythmic agent and the compound of formula (I) are administered within 5 days of each other.

13. The method of claim 12, wherein the antarrhythmic agent and the compound of formula (I) are administered within 24 hours of each other.

14. The method of claim 13, wherein the antarrhythmic agent and the compound of formula (I) are administered within 1 hour of each other.

15. The method of claim 14, wherein the antarrhythmic agent and the compound of formula (I) are administered simultaneously.

16. The method of claim 6, wherein the antiarrhythmic agent is administered in amount between 0.1 mg and 10,000 mg per day.

17. The method of claim 16, wherein the antiarrhythmic agent is administered in amount between 0.1 mg and 1000 mg per day.

18. The method of claim 17, wherein the antiarrhythmic agent is administered in amount between 0.1 mg and 100 mg per day.

19. The method of claim 6, wherein the compound of formula (I) is administered in amount between 0.1 mg and 1000 mg per day.

20. The method of claim 19, wherein the compound of formula (I) is administered in amount between 0.1 mg and 100 mg per day.

21. The method of claim 20, wherein the compound of formula (I) is administered in amount between 0.1 mg and 10 mg per day.

22. The method of claim 6, further comprising the step of administering to said patient one or more additional cancer treatments selected from the group consisting of surgery, radiation, chemotherapy, immunotherapy, anti-angiogenesis therapy, and gene therapy.

23. A kit, comprising:
(i) a composition comprising an antiarrhythmic agent and a compound of formula (I), or a pharmaceutically acceptable salt thereof; and
(ii) instructions for administering said composition to a patient diagnosed with or at risk of developing a neoplasm.

24. A kit, comprising:
(i) an antiarrhythmic agent;
(ii) a compound of formula (I), or a pharmaceutically acceptable salt thereof; and
(iii) instructions for administering said antiarrhythmic agent and said compound of formula (I) to a patient diagnosed with or at risk of developing a neoplasm.

25. A kit, comprising:
(i) an antiarrhythmic agent; and
(ii) instructions for administering said antiarrhythmic agent and a compound of formula (I), or a pharmaceutically acceptable salt thereof, to a patient diagnosed with or at risk of developing a neoplasm.

26. A kit, comprising:
(i) a compound of formula (I), or a pharmaceutically acceptable salt thereof; and
(iii) instructions for administering said compound of formula (I) and an antiarrhythmic agent to a patient diagnosed with or at risk of developing a neoplasm.
